(19) United States
(12) Patent Application Publication
(10) Pub. No.: US 2026/0137816 A1
(43) Pub. Date: May 21, 2026

(54) IMAGING PHANTOM

(71) Applicant: The Regents of the University of Michigan, Ann Arbor, MI (US)

(72) Inventors: Scott D. Swanson, Ann Arbor, MI (US); Dariya Malyarenko, Ann Arbor, MI (US); Thomas L. Chenevert, Ann Arbor, MI (US)

(21) Appl. No.: 19/366,641

(22) Filed: Oct. 23, 2025

Related U.S. Application Data
(63) Continuation of application No. 16/850,273, filed on Apr. 16, 2020.
(60) Provisional application No. 62/835,709, filed on Apr. 18, 2019.

(51) Int. Cl. A61K 49/18; A61L 31/02; A61L 31/18; G01R 33/58

(52) U.S. Cl. CPC A61K 49/1896; A61L 31/028; A61L 31/18; G01R 33/58

(57) ABSTRACT

Provided herein is technology relating to medical imaging and particularly, but not exclusively, to devices, methods, systems, and kits related to a quantitative diffusion imaging phantom.

This application is a continuation of U.S. patent application Ser. No. 16/850,273, filed Apr. 16, 2020, which claims the benefit of U.S. Provisional Patent Application No. 62/835,709, filed Apr. 18, 2019, each of which is incorporated by reference herein in its entirety.

### STATEMENT REGARDING FEDERALLY SPONSORED RESEARCH OR DEVELOPMENT

This invention was made with government support under CA166104 awarded by the National Institutes of Health. The government has certain rights in the invention.

### FIELD

Provided herein is technology relating to medical imaging and particularly, but not exclusively, to devices, methods, systems, and kits related to a diffusion imaging phantom.

### BACKGROUND

Diffusion kurtosis, a measure of deviation from a Gaussian probability distribution function, is of interest in imaging white matter diseases and cancer. The accuracy, precision, and reproducibility of medical imaging would be improved by a suitably designed diffusion kurtosis imaging (DKI) phantom.

### SUMMARY

Provided herein is technology related to a phantom for quantitative DKI based on controlling restricted diffusion fraction by relative size of vesicles and molecular spacing of lamellar liquid crystal (LLC) systems. Accordingly, in some embodiments, the technology is related to a diffusion kurtosis imaging (DKI) phantom comprising lamellar liquid crystal vesicles provided by mixing a high-molecular weight alcohol, an ionic surfactant, and water solutions. In some embodiments, the DKI phantom vesicles are suspended in water. In some embodiments, the DKI phantom is embedded in gel matrix (e.g., agarose or polyethylene glycol (PEG)) or water solution of hydrophilic macromolecules (e.g., polyvinylpyrrolidone (PVP)). In some embodiments, the high-molecular weight alcohol is decyl, stearyl, cetyl, or behenyl alcohol. In some embodiments, the ionic surfactant is cetyltrimethylammonium bromide, behentrimonium chloride, behentrimonium methyl sulfate, or sodium dodecyl sulfate. In some embodiments, the high-molecular weight alcohol and ionic surfactant are present in a ratio of 1:1 to 10:1 weight % (e.g., 1:1, 2:1, 3:1, 4:1, 5:1, 6:1, 7:1, 8:1, 9:1, or 10:1 wt %). In some embodiments, the high-molecular weight alcohol and ionic surfactant are present in a ratio of 2:1 to 5:1 weight %. In some embodiments the phantom comprises a gel matrix that is agarose or PEG based. In some embodiments the water solution contains PVP. In some embodiments, when data is collected at typical ambient room temperatures between 19° C. and 25° C. (e.g., 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, or 25.0° C.), the DKI phantom produces a Kₐₚₚ of between 0.50 and 3.00 (e.g., 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00). In some embodiments, the DKI phantom produces a Kₐₚₚ of between 0.75 and 1.75. In some embodiments, the DKI phantom produces approximately the same Kₐₚₚ over a two- to six-month period (e.g., over 1, 2, 3, 4, 5, 6, 7, 8, 9, 10, 11, 12, 13, 14, 15, 16, 17, 18, 19, 20, 21, 22, 23, 24, or 25 weeks).

In some embodiments, the technology provides a DKI phantom comprising a plurality of vials. In some embodiments, each vial comprises a different lamellar liquid crystal system provided by mixing a high molecular weight alcohol, an ionic surfactant, and water. In some embodiments, the DKI phantom is water based. In some embodiments, the DKI phantom is embedded in gel matrix (e.g., agarose or PEG based) or water solution of macromolecules (e.g., PVP). In some embodiments, each vial comprises decyl, stearyl, cetyl, or behenyl alcohol mixed with cetyltrimethylammonium bromide, behentrimonium chloride, behentrimonium methyl sulfate, or sodium dodecyl sulfate. In some embodiments, the DKI phantom further comprises a negative control vial having a Kₐₚₚ of approximately zero (0), e.g., at 19-25° C.

In some embodiments, the technology relates to methods for validating diffusion kurtosis imaging (DKI) parameters as quantitative imaging biomarkers. For example, in some embodiments, the method comprises providing a DKI phantom comprising a lamellar liquid crystal system provided by mixing a high-molecular weight alcohol, an ionic surfactant, and water (e.g., free or embedded in gel matrix); and determining a Kₐₚₚ value from diffusion weighted imaging data obtained from performing a magnetic resonance imaging scan of said DKI phantom. In some embodiments, performing a magnetic resonance imaging scan at typical ambient room temperatures (e.g., between 19° C. and 25° C. (e.g., 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, or 25.0° C.)) of said DKI phantom comprises using multiple (e.g., 3 or more (e.g., 3, 4, 5, 6, 7, 8, 9, 10, or more than 10)) diffusion weighting b-values with at least one of said b-values at or above 1000 s/mm² (e.g., at least 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, or 5000 s/mm²). In some embodiments, methods further comprise determining Dₐₚₚ and/or Kₐₚₚ. In some embodiments, determining Dₐₚₚ and/or Kₐₚₚ comprises applying DKI fit model constraints (e.g., Dₐₚₚ<D_{water} at phantom temperature and/or bₘₐₓ Dₐₚₚ K—ₐₚₚ<3). In some embodiments, methods further comprise comparing the Kₐₚₚ value to a previously determined Kₐₚₚ value. In some embodiments, methods further comprise comparing the Kₐₚₚ value to a Kₐₚₚ value determined using another magnetic resonance imaging scanner under similar conditions. In some embodiments of methods, the high-molecular weight alcohol is decyl, stearyl, cetyl, or behenyl alcohol. In some embodiments of methods, ionic surfactant is cetyltrimethylammonium bromide, behentrimonium chloride, behentrimonium methyl sulfate, or sodium dodecyl sulfate. In some embodiments of methods, the high-molecular weight alcohol and said ionic surfactant are present in a ratio of 1:1 to 10:1 weight % (e.g., 1:1, 2:1, 3:1, 4:1, 5:1, 6:1, 7:1, 8:1, 9:1, or 10:1 wt %). In some embodiments of methods, the high-molecular weight alcohol and said ionic surfactant are present in a ratio of 2:1 to 5:1 wt %. In some embodiments the phantom comprises a gel matrix that is agarose or polyethylene glycol based. In some embodiments the phantom water solution included hydrophilic macromolecules of PVP. In some embodiments of methods, the DKI phantom produces a Kₐₚₚ of between 0.50 and 3.00 (e.g., 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00). In some embodiments of methods, the DKI phantom produces a Kₐₚₚ of between 0.75 and 1.75. In some embodiments of methods, the DKI phantom produces approximately the same Kₐₚₚ over a two- to six month period (e.g., over 1, 2, 3, 4, 5, 6, 7, 8, 9, 10, 11, 12, 13, 14, 15, 16, 17, 18, 19, 20, 21, 22, 23, 24, and weeks). In some embodiments of methods, the DKI phantom comprises a plurality of vials, wherein each vial comprises vesicles formed by different lamellar liquid crystal system provided by mixing a high-molecular weight alcohol, a surfactant (e.g., an ionic or a nonionic surfactant), and water. In some embodiments, the DKI phantom is water-based. In some embodiments, the DKI phantom is embedded in gel matrix (e.g., agarose or polyethylene glycol based) and macromolecular solutions (e.g., PVP). In some embodiments of methods, the DKI phantom further comprises a negative control vial having a Kₐₚₚ of approximately 0, e.g., at 19-25° C.

In some embodiments, the technology provides a system comprising a DKI phantom as described herein and a first magnetic resonance imaging scanner. In some embodiments of systems, the system further comprises a second magnetic resonance imaging scanner. In some embodiments of systems, the technology is related to a diffusion kurtosis imaging (DKI) phantom comprising vesicles formed by a lamellar liquid crystal system provided by mixing a high-molecular weight alcohol, a surfactant (e.g., an ionic surfactant or a nonionic surfactant), and water. In some embodiments, the DKI phantom is water based. In some embodiments, the DKI phantom is embedded in gel matrix (e.g., agarose or PEG based) or water solutions of macromolecules (e.g., PVP). In some embodiments of systems, the high-molecular weight alcohol is decyl, stearyl, cetyl, or behenyl alcohol. In some embodiments of systems, the ionic surfactant is cetyltrimethylammonium bromide or behentrimonium chloride. In some embodiments of systems, the high-molecular weight alcohol and ionic surfactant are present in a ratio of 1:1 to 10:1 wt % (e.g., 1:1, 2:1, 3:1, 4:1, 5:1, 6:1, 7:1, 8:1, 9:1, or 10:1 wt %). In some embodiments of systems, the high-molecular weight alcohol and ionic surfactant are present in a ratio of 2:1 to 5:1 wt %. In some embodiments of systems, e.g., when data is collected at typical ambient room temperatures between 19° C. and 25° C., the DKI phantom produces a Kₐₚₚ of between 0.50 and 3.00 (e.g., 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00). In some embodiments of systems, the DKI phantom produces a Kₐₚₚ of between 0.75 and 1.75. In some embodiments of systems, the DKI phantom produces approximately the same Kₐₚₚ over a two-month to six-month period (e.g., over 1, 2, 3, 4, 5, 6, 7, 8, 9, 10, 11, 12, 13, 14, 15, 16, 17, 18, 19, 20, 21, 22, 23, 24, and 25 weeks). In some embodiments of systems, the technology provides a DKI phantom comprising a plurality of vials. In some embodiments of systems, each vial comprises vesicle suspensions formed by a different lamellar liquid crystal system provided by mixing a high-molecular weight alcohol, an ionic surfactant, and water. In some embodiments, the DKI phantom is water-based. In some embodiments, the DKI phantom is embedded in gel matrix (e.g., agarose or PEG). In some embodiments of systems, each vial comprises decyl, stearyl, cetyl, or behenyl alcohol mixed with cetyltrimethylammonium bromide, behentrimonium chloride, or sodium dodecyl sulfate. In some embodiments of systems, the DKI phantom further comprises a negative control vial having a Kₐₚₚ of approximately 0. In some embodiments of systems, the system further comprises a component configured to compare a first Kₐₚₚ obtained from the first magnetic imaging scanner and a second Kₐₚₚ obtained from the second magnetic imaging scanner. In some embodiments, the technology provides a system comprising a DKI phantom as described herein and a quantitative analysis software tool. In some embodiments, the system further comprises a second quantitative analysis software tool. In some embodiments, the system further comprises an algorithm configured to compare a first Kₐₚₚ obtained from the first quantitative analysis software tool and a second Kₐₚₚ obtained from the second quantitative analysis software tool.

Some embodiments relate to kits comprising a DKI phantom as described herein. In some embodiments, a diffusion kurtosis imaging (DKI) phantom kit comprises one or more vials comprising vesicular suspensions formed by a lamellar liquid crystal system provided by mixing a high molecular weight alcohol, a surfactant (e.g., an ionic surfactant or a nonionic surfactant), and water. In some embodiments, the DKI phantom is water-based. In some embodiments, the DKI phantom is embedded in gel matrix (e.g., agarose or PEG based) or macromolecule solutions (e.g., PVP). In some embodiments, the high-molecular weight alcohol is decyl, stearyl, cetyl, or behenyl alcohol. In some embodiments, the ionic surfactant is cetyltrimethylammonium bromide, behentrimonium chloride, behentrimonium methyl sulfate, or sodium dodecyl sulfate. In some embodiments, the high-molecular weight alcohol and ionic surfactant are present in a ratio of 1:1 to 10:1 weight % (e.g., 1:1, 2:1, 3:1, 4:1, 5:1, 6:1, 7:1, 8:1, 9:1, or 10:1 wt %). In some embodiments, the high-molecular weight alcohol and ionic surfactant are present in a ratio of 2:1 to 5:1 weight %. In some embodiments, the DKI phantom produces a Kₐₚₚ of between 0.50 and 3.00 (e.g., 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00). In some embodiments, the DKI phantom produces a Kₐₚₚ of between 0.75 and 1.75. In some embodiments, the DKI phantom produces approximately the same Kₐₚₚ over a two- to six-month period (e.g., over 1, 2, 3, 4, 5, 6, 7, 8, 9, 10, 11, 12, 13, 14, 15, 16, 17, 18, 19, 20, 21, 22, 23, 24, and 25). In some kit embodiments, the technology provides a DKI phantom kit comprising a plurality of vials. In some kit embodiments, each vial comprises vesicular suspensions formed by a different lamellar liquid crystal system provided by mixing a high-molecular weight alcohol, an ionic surfactant, and water. In some embodiments, the DKI phantom is water-based. In some embodiments, the DKI phantom is embedded in gel matrix (e.g., agarose or PEG based) or water-macromolecule solutions (e.g., PVP-based). In some kit embodiments, each vial comprises decyl, stearyl, cetyl, or behenyl alcohol mixed with cetyltrimethylammonium bromide, behentrimonium chloride, or sodium dodecyl sulfate. In some kit embodiments, the DKI phantom kit further comprises a negative control vial having a Kₐₚₚ of approximately 0.

In some embodiments, the technology provides use of a DKI phantom as described herein to evaluate and/or calibrate a magnetic resonance imaging scanner. In some embodiments, the technology relates to use of a DKI phantom as described herein to compare a first magnetic resonance imaging scanner to a second magnetic imaging scanner. In some embodiments, the technology provides use of a DKI phantom as described herein to evaluate and/or calibrate quantitative MRI diffusion imaging model fit algorithm accuracy and physical constraints. In some embodiments, the technology relates to use of a DKI phantom as described herein to compare a first algorithm accuracy to a second algorithm accuracy and physical constraints. In some embodiments, a DKI phantom as described herein finds use in evaluating and/or validating a quantitative analysis tool. In some embodiments, a DKI phantom as described herein finds use in comparing a first analysis tool to a second analysis tool.

Additional embodiments will be apparent to persons skilled in the relevant art based on the teachings contained herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features, aspects, and advantages of the present technology will become better understood with regard to the following drawings.

FIG. 1A shows magnetic resonance imaging image data for Dₐₚₚ (left) and Kₐₚₚ (right).

FIG. 1B shows the positions of the seven sample vials in FIG. 1A, left and right.

FIG. 2A is a plot of Kₐₚₚ as a function of Dₐₚₚ.

FIG. 2B is a plot showing the variation in Kₐₚₚ over time.

FIG. 2C is a plot showing the variation in Dₐₚₚ over time.

FIG. 3 is a series of magnetic resonance imaging image data for duplicate phantom samples tested at three different sites.

FIG. 4 is a plot showing the diffusion of water in vesicles and lamellar structures at high b.

FIG. 5 is a plot showing decay of the water signal due to diffusion in phantom vials from b=0 to 3000.

FIG. 6 is a plot showing vesicle sizes.

FIG. 7 is a plot showing diffusion data for vesicles.

FIG. 8A and FIG. 8B provide a schematic two-dimensional visualization of free (colored) and restricted (white) compartments. Doubling the diameter of vesicles with fixed total circumference (or fixed weight percent molecules) doubles the fraction of restricted water molecules. FIG. 8A shows that a collection of 16 circles with a diameter of 0.5 units has a restricted fraction of 25% and FIG. 8B shows that a collection of 8 circles of diameter 1 unit has a restricted fraction of 50%. A congruent trend occurs with surface area and volume in the three-dimensional case.

FIG. 9A is a plot of DLS data measuring vesicle size at CA:CTAB molar ratios of 1:1, 3:1, 4:1, and 5:1. The data indicated that the CA:CTAB molar ratio determines vesicle size.

FIG. 9B is a representative TEM image of a 1.38-micron vesicle (B). The electron microscopy data confirm vesicle shape and dimension.

FIGS. 10A to 10F are plots of DLS and NMR diffusion data for CA:CTAB vesicles. FIG. 10A is a plot of data indicating that vesicle diameter increases linearly from 210 nm to 1680 nm as CA:CTAB molar ratio varies from 1:1 to 6:1. FIG. 10B is a plot of diffusion NMR data at CA:CTAB from 1:1 to 6:1 molar ratio and fitted with a biexponential model. FIG. 10C is a plot of the free diffusion constant, D_{f}, as a function of vesicle diameter in the biexponential model. FIG. 10D is a plot of the restricted diffusion fraction, pᵣ, as a function of vesicle diameter in the biexponential model. FIG. 10E is a plot of Dₐₚₚ as a function of vesicle diameter in the kurtosis model. FIG. 10F is a plot of Kₐₚₚ as a function of vesicle diameter in the kurtosis model.

FIGS. 11A and 11B are plots of data showing that phantom samples comprising an alkyl ammonium surfactant (e.g., dimethyldioctadecylammonium chloride (e.g., commercially available as ARQUAD 2HT-75 from SIGMA)) provide an increased range of Dₐₚₚ values.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

Provided herein is technology relating to medical imaging and particularly, but not exclusively, to devices, methods, systems, and kits related to a diffusion imaging phantom.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control. The section headings used herein are for organizational purposes only and are not to be construed as limiting the described subject matter in any way.

### Definitions

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the terms “about”, “approximately”, “substantially”, and “significantly” are understood by persons of ordinary skill in the art and will vary to some extent on the context in which they are used. If there are uses of these terms that are not clear to persons of ordinary skill in the art given the context in which they are used, “about” and “approximately” mean plus or minus less than or equal to 95% confidence-intervals of the particular term with p-value >0.05 and “substantially” and “significantly” mean plus or minus greater than 95% confidence intervals of the particular term and p-value <0.05.

As used herein, disclosure of ranges includes disclosure of all values and further divided ranges within the entire range, including endpoints and sub-ranges given for the ranges.

As used herein, the suffix “-free” refers to an embodiment of the technology that omits the feature of the base root of the word to which “-free” is appended. That is, the term “X-free” as used herein means “without X”, where X is a feature of the technology omitted in the “X-free” technology. For example, a “calcium-free” composition does not comprise calcium, a “mixing-free” method does not comprise a mixing step, etc.

Although the terms “first”, “second”, “third”, etc. may be used herein to describe various steps, elements, compositions, components, regions, layers, and/or sections, these steps, elements, compositions, components, regions, layers, and/or sections should not be limited by these terms, unless otherwise indicated. These terms are used to distinguish one step, element, composition, component, region, layer, and/or section from another step, element, composition, component, region, layer, and/or section. Terms such as “first”, “second”, and other numerical terms when used herein do not imply a sequence or order unless clearly indicated by the context. Thus, a first step, element, composition, component, region, layer, or section discussed herein could be termed a second step, element, composition, component, region, layer, or section without departing from technology.

As used herein, the term “the DKI phantom is water-based” means that the DKI phantom does not comprise a gel or macromolecular solution. While phantom materials may produce a phantom that is viscous, a DKI phantom that is water-based does not comprise materials added to produce a gel-based phantom or water-soluble macromolecules.

The parameter Kₐₚₚ is a unitless parameter. The parameter Dₐₚₚ has units of mm²/s. Where reported as a number without a power of ten, it is to be understood to append×10⁻³ to the reported value of Dₐₚₚ. Dₐₚₚ and Kₐₚₚ ranges are given for phantoms scanned at scanner ambient temperature (see, e.g., Malyarenko (2019) “Multicenter Repeatability Study of a Novel Quantitative Diffusion Kurtosis Imaging Phantom” Tomography 5 (1): 36-43, incorporated herein by reference). Different parameter ranges can be achieved by changing phantom temperature. Dₐₚₚ and Kₐₚₚ values are reported herein at room temperature, e.g., at approximately 19-25° C., unless indicated otherwise.

### Description

Provided herein is technology relating to medical imaging and particularly, but not exclusively, to devices, methods, systems, and kits related to a quantitative diffusion kurtosis imaging (DKI) phantom. The technology described herein provides diffusion-weighted imaging (DWI) acquisition and modeling methods to address non-Gaussian (restricted) water diffusion effects through DKI imaging and modeling. Accordingly, the technology provides more specific measures of tissue structure and biology.

The technology finds use in providing and validating DKI parameters as quantitative imaging biomarkers of tumor response to therapy. In particular, the technology finds use in multicenter oncology trials to improve the precision (repeatability) and accuracy (bias) of the QIBs across multiple scanner platforms (e.g., at different sites) and model fit algorithms using a common scan protocol. In some embodiments, the phantom technology provides true parameter values in physiologically relevant ranges. In some embodiments, the phantom technology finds use in a protocol to estimate bias from scanner to scanner. In some embodiments, the technology relates to use of true phantom parameters to refine fit algorithm accuracy and validate quantitative diffusion model.

During the development of embodiments of the technology, experiments were conducted at typical ambient room temperatures between 19° C. and 25° C. (e.g., 19.0, 19.1, 19.2, 19.3, 19.4, 19.5, 19.6, 19.7, 19.8, 19.9, 20.0, 20.1, 20.2, 20.3, 20.4, 20.5, 20.6, 20.7, 20.8, 20.9, 21.0, 21.1, 21.2, 21.3, 21.4, 21.5, 21.6, 21.7, 21.8, 21.9, 22.0, 22.1, 22.2, 22.3, 22.4, 22.5, 22.6, 22.7, 22.8, 22.9, 23.0, 23.1, 23.2, 23.3, 23.4, 23.5, 23.6, 23.7, 23.8, 23.9, 24.0, 24.1, 24.2, 24.3, 24.4, 24.5, 24.6, 24.7, 24.8, 24.9, or 25.0° C.) to design and fabricate a stable (e.g., temporally stable and/or temperature-stable) quantitative DKI phantom with a range of Dₐₚₚ and Kₐₚₚ values (e.g., in the biologically relevant range). In some embodiments, Dₐₚₚ is in the biologically relevant range (e.g., Dₐₚₚ is 0.2 to 2.0×10⁻³ mm²/s (e.g., 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0)). In some embodiments, Kₐₚₚ is in the biologically relevant range (e.g., Kₐₚₚ is 0.2 to 3.00 (e.g., 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00)). In some embodiments, both Dₐₚₚ and Kₐₚₚ are in the biologically relevant range.

Some of the phantom samples have low viscosity and comprise regions of freely diffusing water and water restricted in multilamellar lipid vesicles. As the b-value increases, the signal from the freely diffusing water is preferentially suppressed, thus increasing the contribution to the signal from water molecules with restricted motions. Other phantom samples are viscous and form areas of randomly oriented lamella and, in general, have slow diffusion and high kurtosis. Ionic surfactants (e.g., CTAB and BTAC) serve two purposes: creating increased lamellar spacing and providing antibiotic activity to slow or eliminate bacterial or other biological degradation. In some embodiments, adjusting the concentrations of the alcohols and/or surfactants provides for tuning the phantoms to provide any clinically relevant kurtosis value.

The phantom provided herein is advantageous. The phantom is easily constructed because it comprises readily available materials and is prepared using a reproducible procedure. The phantom has stable apparent diffusion and kurtosis constants (Dₐₚₚ and Kₐₚₚ, respectively) that are substantially constant as a function of time (e.g., over 1, 2, 3, 4, 5, 6, 7, 8, 9, 10 weeks or more than 10 weeks). The phantom has parameters that are adjustable by changing the composition of the phantom ingredients to tune the diffusion and kurtosis coefficient values to reflect relevant biological ranges for those values. The phantom is isotropic, e.g., the fractional anisotropy values are close to zero.

Further, data indicated that lamellar vesicles provide a system for a quantitative DKI phantom because they have many desirable properties. First, they can be assembled from relatively inexpensive and stable materials. Second, the NMR T2 time of all samples is greater than 500 ms (relaxivity parameter r₂ in CA-BTAC was determined to be 0.276 1/(s-wt %), thus providing high signal to noise ration over a broad b-value range and echo times TE. Third, the ionic surfactants impart charge to the vesicles (a large zeta potential) so that the particles repel each other and stay suspended. Furthermore, the value of Kₐₚₚ can be easily tuned over the range found in organs, tissues, and cells (e.g., in cancer tissues). In addition to creating tunable kurtosis parameters, sample stability is of paramount importance for quantitative MRI phantoms. Data collected from a 6-month study of CA-BTAC indicated that the combination of low solid concentrations and charged ionic vesicles resulted in a stable preparation provided the samples remain near room temperature.

The technology provided herein relates to vesicles and/or lamellar systems in water that provide DKI phantoms. In some embodiments, the phantoms comprise water-soluble macromolecules that modify the physical properties of aqueous systems (e.g., by gelation, thickening, emulsification, and/or stabilization) and thus provide control of water diffusion in the phantoms. In some embodiments, the phantoms comprise macromolecules that are organic molecules such as, e.g., polysaccharides, nucleic acids, and proteins. In some embodiments, the phantoms comprise macromolecules that are natural water soluble polymers such as, e.g., xanthan gum, pectin, chitosan, dextran, carrageenan, guar gum, alginate, cellulose, cellulose ethers (e.g., hydroxypropylmethyl cellulose (HPMC), hydroxypropyl cellulose (HPC), hydroxyethyl cellulose (HEC), sodium carboxy methyl cellulose (Na-CMC)), hyaluronic acid, albumin, starch, starch derivatives, gelatin, hydrolyzed keratin, hydrolyzed silk protein, and the like. In some embodiments, the phantoms comprise macromolecules that are synthetic molecules such as, e.g., polyethylene glycol (PEG), polyvinyl alcohol (PVA), polyacrylic acid (PAA), polyacrylamide, polyvinylpyrrolidone (PVP), N-(2-hydroxypropyl) methacrylamide (HPMA), divinyl ether-maleic anhydride (DIVEMA), polyoxazoline, polyphosphate, polyphosphazene, and the like. See, e.g., Kadajji (2011) “Water Soluble Polymers for Pharmaceutical Applications” Polymers 3 (4): 1972-2009, incorporated herein by reference. In some embodiments, the vesicles comprise phospholipids. In some embodiments, the vesicles comprise lamellar systems comprising long-chain alcohols and surfactants.

In some embodiments, the vesicles comprise quaternary amine compounds (QAC (e.g., a compound having chemical structure NR₄⁺, where each R is independently an alkyl group or an aryl group and the R groups may be the same or different)). See, e.g., IUPAC. Compendium of Chemical Terminology, 2nd ed. (the “Gold Book”), compiled by McNaught and Wilkinson (Blackwell Scientific Publications, Oxford (1997)); Pure & Appl. Chemistry 67:1307-75 (1995), each of which is incorporated herein by reference. In some embodiments, the QAC comprises four R groups each independently comprising from 1 to carbons (e.g., the R groups independently comprise a C1, C2, C3, C4, C5, C6, C7, C8, C9, C10, C11, C12, C13, C14, C15, C16, C17, C18, C19, or C20 alkyl chain). Exemplary QAC include, e.g., 1,2-dioleoyl-3-trimethylammonium-propane (DOTAP), N-[1-(2,3-dioleoyloxy) propyl]-N,N,N-trimethylammonium chloride (DOTMA), 1-[2-(oleoyloxy)ethyl]-2-oleyl-3-(2-hydroxyethyl) imidazolinium chloride (DOTIM), 2,3-dioleyloxy-N-[2 (sperminecarboxamido)ethyl]-N,N-dimethyl-1-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxyethyl ammonium bromide (DMRIE), N-(1,2-dioleoyloxyprop-3-yl)-N,N-dimethyl-N-hydroxyethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), and 1-palmitoyl-2-oleoyl-sn-glycero-3-ethylphosphocholine (16:0-18:1 EPC).

With respect to phospholipids, any lipids that form vesicles may be used. In some embodiments, lipids comprise one or more different lipids. For instance, the lipids can contain up to 100 lipids. The lipids preferably contain 1 to 10 lipids. The lipids may comprise naturally occurring lipids and/or artificial lipids. In particular embodiments, the technology comprises use of ionic (e.g., positively charged or negatively charged) lipids, e.g., comprising a charged head group as described herein. The lipids typically comprise a head group, an interfacial moiety, and two hydrophobic tail groups which may be the same or different. Suitable head groups include, but are not limited to, neutral head groups, such as diacylglycerides (DG) and ceramides (CM); zwitterionic head groups, such as phosphatidyl choline (PC), phosphatidylethanolamine (PE) and sphingomyelin (SM); negatively charged head groups, such as phosphatidylglycerol (PG); phosphatidylserine (PS), phosphatidylinositol (PI), phosphatic acid (PA) and cardiolipin (CA); and positively charged headgroups, such as trimethylammonium-propane (TAP). Suitable interfacial moieties include, but are not limited to, naturally occurring interfacial moieties, such as glycerol-based or ceramide-based moieties. Suitable hydrophobic tail groups include, but are not limited to, saturated hydrocarbon chains, such as lauric acid (n-dodecanolic acid), myristic acid (n-tetradecononic acid), palmitic acid (n-hexadecanoic acid), stearic acid (n-octadecanoic) and arachidic (n-eieosanoic); unsaturated hydrocarbon chains, such as oleic acid (cis-9-octadecanoic); and branched hydrocarbon chains, such as phytanoyl. In some embodiments, the length of the chain and the position and number of the double bonds in the unsaturated hydrocarbon chains vary. The length of the chains and the position and number of the branches, such as methyl groups, in the branched hydrocarbon chains can vary. The hydrophobic tail groups can be linked to the interfacial moiety as an ether or an ester. For instance, exemplary non-limiting phospholipids include, e.g., 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1,2-di-O)-octadecenyl-sn-glycero-3-phosphocholine (18:0 Diether PC), 1,2-dilinolenoyl-sn-glycero-3-phosphocholine (DLnPC), 1,2-diarachidonoyl-sn-glycero-3-phosphocholine (DAPC), 1,2-didocosahexaenoyl-sn-glycero-3-phosphocholine (DHAPC), 1,2-dioleoyl-sn-glycero-3-phosphoethanolamine (DOPE), 1,2-diphytanoyl-sn-glycero-3-phosphoethanolamine (4ME 16:0 PE), 1,2-distearoyl-sn-glycero-3-phosphoethanolamine (DSPE), 1,2-dilinoleoyl-sn-glycero-3-phosphoethanolamine (DLPE), 1,2-dilinolenoyl-sn-glycero-3-phosphoethanolamine (DLnPE), 1,2-diarachidonoyl-sn-glycero-3-phosphoethanolamine (DAPE), 1,2-didocosahexaenoyl-sn-glycero-3-phosphoethanolamine (DHAPE), 1,2-dioleoyl-sn-glycero-3-phospho-rac-(1-glycerol) sodium salt (DOPG), 1-myristoyl-2-palmitoyl-sn-glycero-3-phosphocholine (14:0-16:0 PC, MPPC), 1-myristoyl-2-stearoyl-sn-glycero-3-phosphocholine (14:0-18:0 PC, MSPC), 1-palmitoyl-2-myristoyl-sn-glycero-3-phosphocholine (16:0-14:0 PC, PMPC), 1-palmitoyl-2-stearoyl-sn-glycero-3-phosphocholine (16:0-18:0 PC, PSPC), 1-stearoyl-2-myristoyl-sn-glycero-3-phosphocholine (18:0-14:0 PC, SMPC), or 1-stearoyl-2-palmitoyl-sn-glycero-3-phosphocholine (18:0-16:0 PC, SPPC).

In some embodiments, the lipids are chemically-modified. The head group or the tail group of the lipids may be chemically-modified. Suitable lipids whose head groups have been chemically-modified include, but are not limited to, PEG-modified lipids, such as 1,2-diacyl-sn-glycero 3-phosphoethanolamine-N-[methoxy (polyethylene glycol)-2000]; functionionalized PEG lipids, such as 1,2-distearoyl-sn-glycero-3 phosphoethanolamine-N-[biotinyl(polyethylene glycol) 2000]. Suitable lipids whose tail groups have been chemically-modified include, but are not limited to, polymerizable lipids, such as 1,2-bis(10,12-tricosadiynoyl)-sn-glycero-3-phosphocholine; fluorinated lipids, such as 1-palmitoyl-2-(16-fluoropalmitoyl)-sn-glycero-3-phosphocholine; deuterated lipids, such as 1,2-dipalmitoyl-D6 2-sn-glycero-3-phosphocholine; and ether linked lipids, such as 1,2-di-O)-phytanyl-sn-glycero-3-phosphocholine. In some embodiments, lipids comprise one or more additives that affect the properties of the vesicles. Suitable additives include, but are not limited to, fatty acids, such as palmitic acid, myristic acid and oleic acid; fatty alcohols, such as palmitic alcohol, myristic alcohol and oleic alcohol; sterols, such as cholesterol, ergosterol, lanosterol, sitosterol and stigmasterol; lysophospholipids, such as 1-acyl-2-hydroxy-sn-glycero-3-phosphocholine; and ceramides. The lipid preferably comprises cholesterol and/or ergosterol when membrane proteins are to be inserted into the lipid bilayer.

In some embodiments, the phantoms comprise water soluble macromolecules at a concentration of 0.25% by weight to 20% by weight (e.g., 0.25, 0.50, 0.75, 1.00, 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50, 9.75, 10.00, 10.25, 10.50, 10.75, 11.00, 11.25, 11.50, 11.75, 12.00, 12.25, 12.50, 12.75, 13.00, 13.25, 13.50, 13.75, 14.00, 14.25, 14.50, 14.75, 15.00, 15.25, 15.50, 15.75, 16.00, 16.25, 16.50, 16.75, 17.00, 17.25, 17.50, 17.75, 18.00, 18.25, 18.50, 18.75, 19.00, 19.25, 19.50, 19.75, or 20.00% by weight). In some embodiments, the phantoms comprise vesicle-forming components at a concentration of 0.25% by weight to 20% by weight (e.g., 0.25, 0.50, 0.75, 1.00, 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50, 9.75, 10.00, 10.25, 10.50, 10.75, 11.00, 11.25, 11.50, 11.75, 12.00, 12.25, 12.50, 12.75, 13.00, 13.25, 13.50, 13.75, 14.00, 14.25, 14.50, 14.75, 15.00, 15.25, 15.50, 15.75, 16.00, 16.25, 16.50, 16.75, 17.00, 17.25, 17.50, 17.75, 18.00, 18.25, 18.50, 18.75, 19.00, 19.25, 19.50, 19.75, or 20.00% by weight).

According to embodiments of the technology, the DKI phantoms described herein comprise vesicles having a diameter greater than 200 nm (e.g., greater than 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500 nm), e.g., as measured by light scattering. In some embodiments, the DKI phantoms described herein comprise water soluble macromolecules less than 10 nm in diameter (e.g., less than 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 nm in diameter), e.g., as measured by light scattering. In some embodiments, the water-soluble macromolecules form complexes, aggregates, and/or particles that are less than 10 nm in diameter (e.g., less than 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 nm in diameter), e.g., as measured by light scattering. In some embodiments, the macromolecule has characteristics of a 5-nm particle in solution (e.g., the macromolecule behaves like a particle having a diameter of approximately 1 to 10 nm (e.g., 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 nm)). In some embodiments, the macromolecule interacts with a vesicle (e.g., a vesicle comprising a charge) to provide a vesicle-macromolecule complex. In some embodiments, charge charge interactions, van der Waals, and/or hydrophobic interactions mediate the interaction between a vesicle and a macromolecule.

In some embodiments, the DKI phantoms have Dₐₚₚ and Kₐₚₚ values in the biologically relevant range. In some embodiments, Dₐₚₚ is in the biologically relevant range (e.g., Dₐₚₚ is 0.2 to 2.0×10⁻³ mm²/s (e.g., 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0)). In some embodiments, Kₐₚₚ is in the biologically relevant range (e.g., Kₐₚₚ is 0.2 to 3.0 (e.g., 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00)). In some embodiments, both Dₐₚₚ and Kₐₚₚ are in the biologically relevant range. In some embodiments, phantoms comprise vesicle-forming components from 0.25% to 5.0% (e.g., 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, 3.00, 3.05, 3.10, 3.15, 3.20, 3.25, 3.30, 3.35, 3.40, 3.45, 3.50, 3.55, 3.60, 3.65, 3.70, 3.75, 3.80, 3.85, 3.90, 3.95, 4.00, 4.05, 4.10, 4.15, 4.20, 4.25, 4.30, 4.35, 4.40, 4.45, 4.50, 4.55, 4.60, 4.65, 4.70, 4.75, 4.80, 4.85, 4.90, 4.95, or 5.00%) that provide Dₐₚₚ and Kₐₚₚ in a biologically relevant range. In some embodiments, phantoms comprise water-soluble macromolecules at a concentration of 0.25% by weight to 20% by weight (e.g., 0.25, 0.50, 0.75, 1.00, 1.25, 1.50, 1.75, 2.00, 2.25, 2.50, 2.75, 3.00, 3.25, 3.50, 3.75, 4.00, 4.25, 4.50, 4.75, 5.00, 5.25, 5.50, 5.75, 6.00, 6.25, 6.50, 6.75, 7.00, 7.25, 7.50, 7.75, 8.00, 8.25, 8.50, 8.75, 9.00, 9.25, 9.50, 9.75, 10.00, 10.25, 10.50, 10.75, 11.00, 11.25, 11.50, 11.75, 12.00, 12.25, 12.50, 12.75, 13.00, 13.25, 13.50, 13.75, 14.00, 14.25, 14.50, 14.75, 15.00, 15.25, 15.50, 15.75, 16.00, 16.25, 16.50, 16.75, 17.00, 17.25, 17.50, 17.75, 18.00, 18.25, 18.50, 18.75, 19.00, 19.25, 19.50, 19.75, or 20.00% by weight) to control and/or provide Dₐₚₚ in a biologically relevant range.

In some embodiments, the phantoms have Dₐₚₚ values between 0.2 and 2.2×10⁻³ mm²/s (e.g., 0.002, 0.004, 0.006, 0.008, 0.010, 0.012, 0.014, 0.016, 0.018, 0.020, 0.022, 0.024, 0.026, 0.028, 0.030, 0.032, 0.034, 0.036, 0.038, 0.040, 0.042, 0.044, 0.046, 0.048, 0.050, 0.052, 0.054, 0.056, 0.058, 0.060, 0.062, 0.064, 0.066, 0.068, 0.070, 0.072, 0.074, 0.076, 0.078, 0.080, 0.082, 0.084, 0.086, 0.088, 0.090, 0.092, 0.094, 0.096, 0.098, 0.100, 0.102, 0.104, 0.106, 0.108, 0.110, 0.112, 0.114, 0.116, 0.118, 0.120, 0.122, 0.124, 0.126, 0.128, 0.130, 0.132, 0.134, 0.136, 0.138, 0.140, 0.142, 0.144, 0.146, 0.148, 0.150, 0.152, 0.154, 0.156, 0.158, 0.160, 0.162, 0.164, 0.166, 0.168, 0.170, 0.172, 0.174, 0.176, 0.178, 0.180, 0.182, 0.184, 0.186, 0.188, 0.190, 0.192, 0.194, 0.196, 0.198, or 0.200 mm²/s). In some embodiments, the phantoms have an upper limit for Dₐₚₚ that is approximately the self-diffusion coefficient of free water, e.g., approximately 3.0×10⁻⁹ m²/s at 37° C. In some embodiments, the upper limit is corrected for a phantom at room temperature (e.g., 19-25° C.).

In some embodiments, the phantoms have Kₐₚₚ values between 0 (e.g., pure water) and 3.0 (e.g., the upper bound for some tissues (e.g., brain tissue, cancer tissue)) (e.g., 0.00, 0.05, 0.10, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60, 2.65, 2.70, 2.75, 2.80, 2.85, 2.90, 2.95, or 3.00).

Embodiments of the technology provide systems. For example, some embodiments of the technology provided herein further comprise functionalities for collecting, storing, and/or analyzing data. For example, in some embodiments the technology comprises a device comprising a processor, a memory, and/or a database for, e.g., storing and executing instructions, analyzing data, performing calculations using the data, transforming the data, and storing the data. Moreover, in some embodiments a processor is configured to control a device such as a magnetic resonance scanner. In some embodiments, the processor is used to initiate and/or terminate the measurement and data collection. In some embodiments, the device comprises a user interface (e.g., a keyboard, buttons, dials, switches, and the like) for receiving user input that is used by the processor to direct a measurement. In some embodiments, the device further comprises a data output for transmitting data to an external destination, e.g., a computer, a display, a network, and/or an external storage medium. In some embodiments, a computer is configured for obtaining, recording, evaluating, and/or comparing one or more non-Gaussian diffusion metric values determined using a phantom and quantitative diffusion model as described herein.

Although the disclosure herein refers to certain illustrated embodiments, it is to be understood that these embodiments are presented by way of example and not by way of limitation.

### EXAMPLES

### Example 1

Diffusion kurtosis arises from impeded Brownian motion created by cell walls. During the development of embodiments of the technology provided herein, experiments were conducted to design, fabricate, and test a series of imaging phantoms that mimic biological materials (e.g., tissues, organs, and/or cells) that impede free water diffusion. The imaging phantoms comprise vesicles formed by lamellar liquid crystal (LLC) systems with molecular mesostructures. In some embodiments, the molecular mesostructures are similar to nerve tissues. The phantom comprises a number (e.g., 7) of samples comprising a mixture of a 1) high-molecular weight alcohol (e.g., decyl, stearyl, cetyl, or behenyl alcohols); 2) an ionic surfactant (e.g., cetyltrimethylammonium bromide (CTAB), behentrimonium chloride (BTAC), or sodium dodecyl sulfate (SDS)); and 3) water. Test vials were prepared as follows:

Vial 4 (2% CTAB and 2% cetearyl alcohol): 600 ml of deionized water was heated to boiling for 5 minutes and cooled to 90° C. 12 g of Hexadecyltrimethylammonium bromide (CTAB, CAS 57-09-0) was dissolved in the hot water. 12 g of SZ cetearyl alcohol (50/50 cetyl alcohol/stearyl alcohol) (CAS #8038-54-8) was melted on a hot plate and poured into the hot CTAB solution. The heating was stopped and mixture was stirred for approximately 10 minutes before pouring it into three 50-ml centrifuge tubes and allowed to cool.

Vial 5 (2% Prolipid 161): 600 ml of deionized water was heated to boiling for 5 minutes and cooled to 90° C. 12 g of Prolipid 161 (PL161, Ashland, comprising cetearyl alcohol, behenyl alcohol, and hydroxyethyl cetearamidopropyldimonium chloride (e.g., a mixture of CAS 67762-27-0, 243982-96-9, 39669-97-1, 7651-02-7, and 1150297 (product code 831030)) was melted in a small beaker on a hotplate, poured into the hot water, and stirred with a magnet. Approximately 1 g of PL161 on the beaker wall was scraped with spatula and added to the hot water. Residual PL161 on the small beaker was transferred by pipetting the hot mixture into the small beaker, melting/dissolving the residual PL161, and returning the solution to the hot water/PL161 mixture. The mixture was stirred for approximately 10 minutes at 85° C. and then poured into three 50-ml centrifuge tubes and allowed to cool.

Vial 6 (CTAB/decanol/water (9/10/81 wt %)): 165 ml of deionized water was heated to boiling for 5 minutes, cooled to 90° C., and added to a beaker. 18.9 g CTAB was dissolved in the hot water. 19.5 g of n-decyl alcohol (decanol) (CAS 112-31-1), was added to the hot water with active stirring. The mixture was immediately viscous and gloopy. The gloopy mixture was poured and scraped into 50-ml centrifuge tubes. Each sample tube was centrifuged for 3 minutes at 2400 rpm to remove air bubbles.

Vial 7 (diluted Tresemme, termed CA-BTAC): 350 ml of deionized water was heated to boiling for 5 minutes, cooled to 90° C., and added to a large Erlenmeyer flask. 50 ml of Tresemme Healthy Volume (UPC 0 22400 39372 8, stamp 122116CA05) was heated to 85° C. and added to the hot water. The mixture was stirred for 10 minutes at 80° C. and poured into three 50-ml centrifuge tubes and allowed to cool. Tresemme comprises approximately 8.3% w/w solids/water. The final phantom sample comprises approximately 1% w/w solids.

Vials of pure water (vial 1), water comprising 20% polyvinylpyrrolidone (PVP) (vial 2), and water comprising 40% PVP (vial 3) were used as negative controls for kurtosis. The Kₐₚₚ for negative controls was expected to be zero or to be substantially or effectively zero. Three vials of each of the 7 phantom vials were studied over a four-week period with a total of 9 measurements to assess stability of the phantom.

All seven phantom materials were individually housed in polypropylene vials (V1-V7) of 150 mm in length and 25 mm of diameter, in a circular arrangement, submerged in water bath in a 1-L plastic jar. Three identical phantom prototypes were prepared using the same material batch, labeled for consistent scan geometry, and shipped to each of the participating sites. The jars were filled with tap water on-site and scanned at ambient temperature.

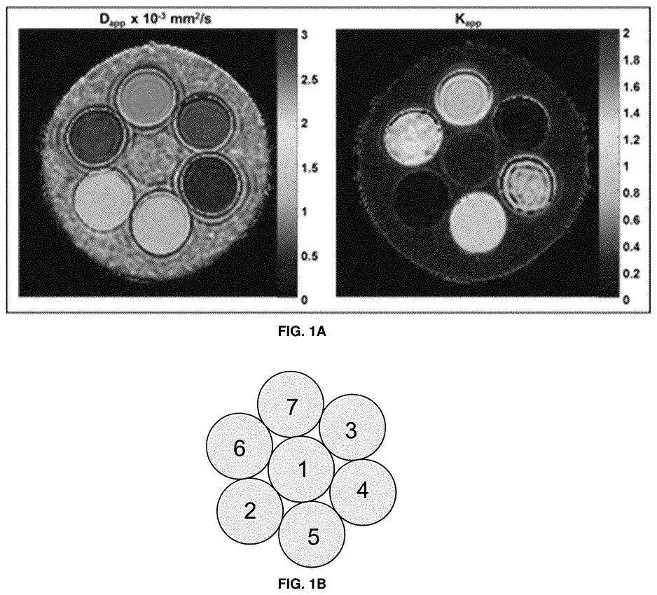

Diffusion Weighted Imaging (DWI) data for the phantom were acquired using 3T clinical MRI scanners using a single-shot DW-EPI sequence with eleven b-values (0, 50, 100, 200, 500, 800, 1000, 1500, 2000, 2500, and 3000 s/mm³). MR parameters were: TR=10 s, TE=105 ms, field of view (FOV) 22 cm², matrix size 128×128, and 3-5 slices. Apparent diffusion and Kurtosis coefficient values were estimated by fitting DWI data as a function of b using the following equation:

$$ln [ S ( b ) / S ( 0 ) ] = - b {D}_{a p p} + \frac{1}{6} {{K}_{a p p} ( b {D}_{a p p} )}^{2} \qquad ( 1 )$$

where S (b) is the signal intensity at the echo time, Dₐₚₚ is the apparent diffusion coefficient, and Kₐₚₚ is the apparent diffusional kurtosis (see, e.g., Jensen and Helpern (2003) “Quantifying non-Gaussian water diffusion by means of pulsed-field gradient MRI”, In Proceedings of the 11th Annual Meeting of ISMRM, Toronto, Canada, p 2154; Jensen et al. (2005) “Diffusional Kurtosis Imaging: The Quantification of Non-Gaussian Water Diffusion by Means of Magnetic Resonance Imaging” Magnetic Resonance in Medicine 53:1432-40, each of which is incorporated herein by reference). The parameter b is given by the usual expression

$$b = {( γ δ g )}^{2} ( Δ - \frac{δ}{3} ) \qquad ( 2 )$$

where γ is the proton gyromagnetic ratio. The Kurtosis model converges when Dₐₚₚ Kₐₚₚb<3, so fit regions of the data are limited to a bₘₐₓ to satisfy this criterion. In addition, at higher b-values the water signal approaches the noise floor and needs to be excluded to avoid Rician bias. The estimated phantom Dₐₚₚ (10⁻³ mm²s⁻¹) and Kₐₚₚ results for the phantom are shown in Table 1. Images are shown in FIG. 1A and FIG. 1B shows the vial placement for FIG. 1A. No appreciable kurtosis was detected in vials comprising control samples 1 (pure water), 2 (20% PVP), and 3 (40% PVP). Kurtosis values between 0.75 and 1.67 were observed for vial 4 (comprising cetearyl alcohol, CTAB, and water), vial 5 (comprising 2% Prolipid161), vial 6 (comprising decanol, CTAB, and water), and vial 7 (comprising cetearyl alcohol, BTAC, and water plus other ingredients). The coefficient of variance for the 9 measurements for each of the 7 samples was less than 5% of the mean.

TABLE 1
Dₐₚₚ and Kₐₚₚ for control and test phantom vials

| Sample | Dₐₚₚ(10⁻³) | Kₐₚₚ | bₘₐₓ |
|---|---|---|---|
| 1. Water | 2.13 +/− 0.03 | 0.02 +/− 0.01 | 2000 |
| 2. 20% PVP | 1.27 +/− 0.016 | 0.01 +/− 0.01 | 3000 |
| 3. 40% PVP | 0.6 +/− 0.006 | 0.01 +/− 0.01 | 3000 |
| 4. CA-CTAB | 0.385 +/− 0.004 | 0.75 +/− 0.06 | 3000 |
| 5. PL161 | 1.13 +/− 0.01 | 1.15 +/− 0.03 | 2000 |
| 6. DEC-CTAB | 0.57 +/− 0.006 | 1.22 +/− 0.05 | 3000 |
| 7. CA-BTAC | 1.045 +/− 0.013 | 1.67 +/− 0.07 | 1500 |

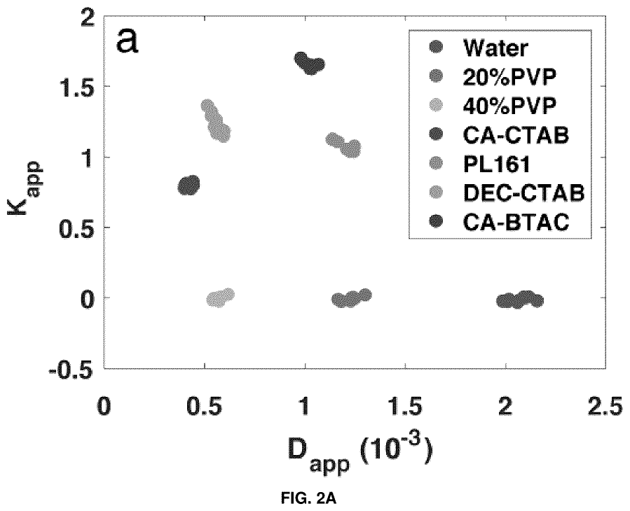

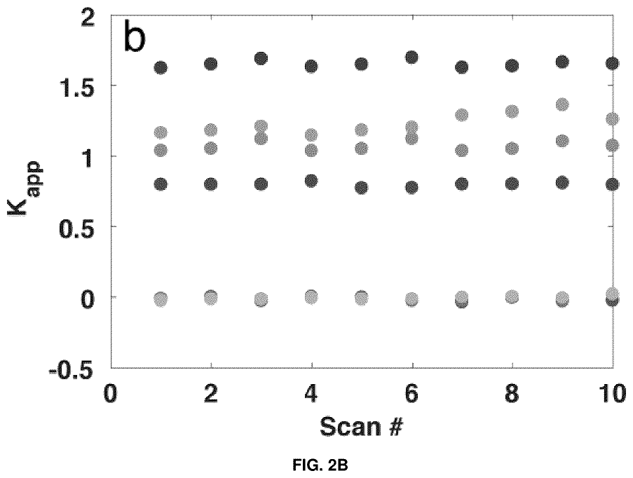

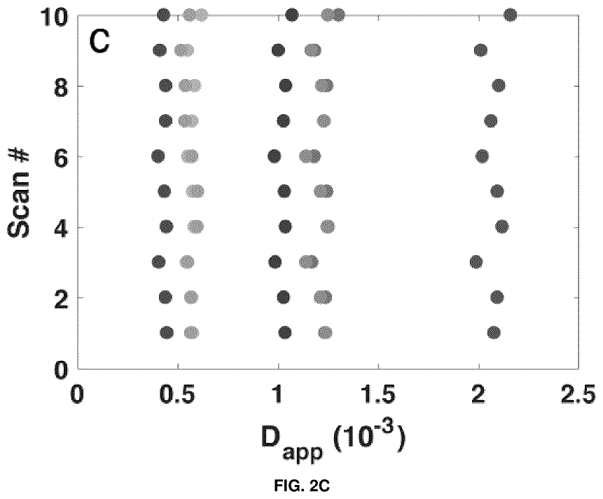

During the development of embodiments of the technology, the thermal stability of the phantom samples was evaluated by measuring Kₐₚₚ and Dₐₚₚ as a function of time. 10 scans were performed over a 2-month period and Kₐₚₚ and Dₐₚₚ parameters were estimated (FIG. 2). The variation in Kₐₚₚ (b), Dₐₚₚ (c), and Kₐₚₚ as a function of Dₐₚₚ (a) are shown in FIG. 2. Variation in Kₐₚₚ is related to changes in phantom temperature as indicated by the decrease in Kₐₚₚ as Dₐₚₚ increases (FIG. 2a), coherent with non-kurtotic controls (water and PVP).

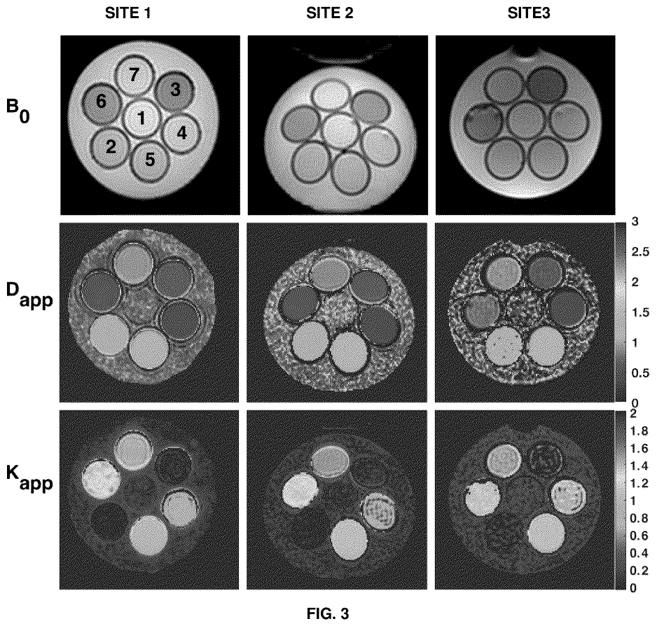

During the development of embodiments of the technology, multisite studies were conducted to evaluate the performance of the phantom at different sites (e.g., using the same imaging protocol at different sites). Three identical phantoms were made at a first site and studied with the same imaging protocol at the first site and two other sites. Specifically, Diffusion Weighted Imaging (DWI) data for the phantom were acquired on 3T clinical MRI scanners using single-shot DW-EPI sequence with eleven b-values. MR parameters were: TR=10 s, TE=105 ms, field of view (FOV) 22 cm², matrix size 128×128, 3-5 slices. Studies were performed at ambient room temperature. Analysis of the data indicated agreement for Dₐₚₚ and Kₐₚₚ at the three sites (FIG. 3).

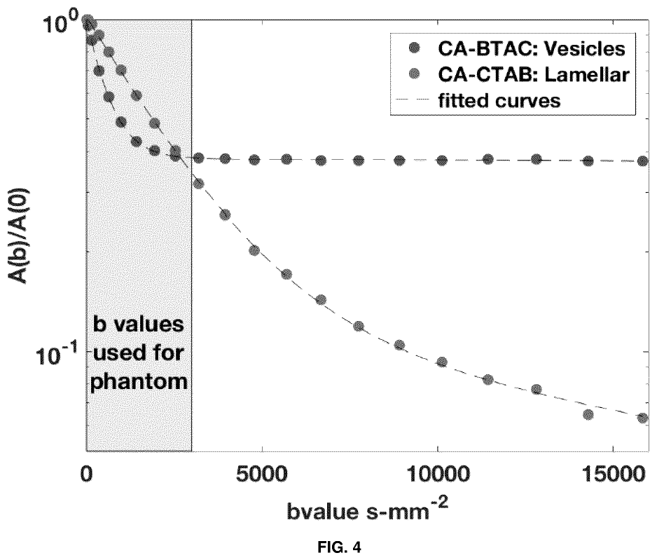

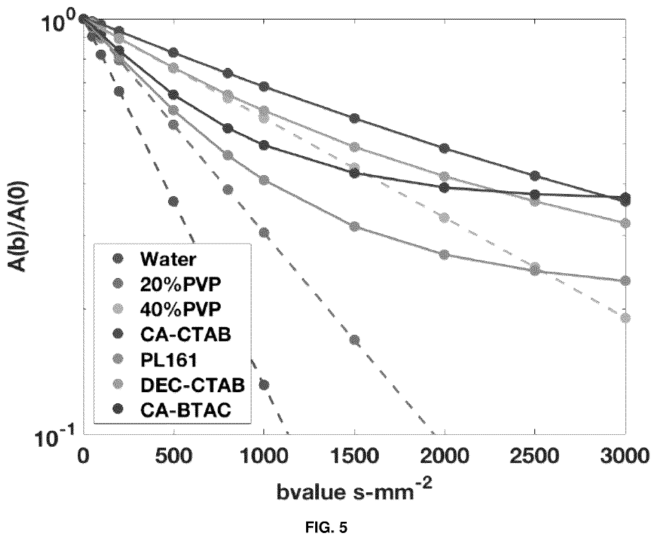

During the development of embodiments of the technology described herein, experiments were conducted to evaluate the diffusive behavior of compositions provided as phantom samples herein. FIG. 4 shows the diffusion of water in vesicles and lamellar structures at high b. Vesicles of CA-BTAC (vial 7) create regions of freely diffusing water and highly restricted diffusion leading to an NMR signal plateau at high b (FIG. 4, upper line). Lamellar structures in CA-CTAB (vial 4) generate few regions of free diffusion, but also regions of moderately restricted diffusion (FIG. 4, lower line). Both systems are well modeled by two compartments with different pool sizes and diffusion constants and fitted lines are shown. FIG. 5 shows decay of the water signal due to diffusion in all phantom vials from b=0 to 3000. Water, 20% PVP, and 40% PVP (dotted lines from left to right, respectively) show monotonic decay with increasing b values (Kurtosis=0). Alcohol-surfactant systems show variable diffusion and kurtosis.

Recent studies show that Diffusion Kurtosis Imaging (DKI) may be useful in characterizing and monitoring changes in cellularity associated with cancer progression and therapy response. These studies highlight the need for quantitative DKI phantoms that validate results in multisite clinical settings. The synthetic DKI phantoms developed to date lack range of diffusion parameters observed in vivo. Experiments were conducted to provide tunable nanostructured materials (e.g., for use in phantoms as described herein) that generate diffusion and kurtosis values observed in vivo. These nanostructures provide a platform for a quantitative diffusion kurtosis phantom. During the development of embodiments of the technology provided herein, experiments were conducted to test the changes in vesicle size and diffusion behavior for phantom samples as a function of sonication. Diffusion of water is related to vesicle size because the restricted diffusion fraction is determined by the volume fraction of water molecules encapsulated in vesicles. For a given amount of surface area (constant mole fraction of vesicle forming molecules), the volume fraction is proportional to the diameter of the vesicles. A large number of small vesicles will have a smaller volume fraction that a smaller number of larger vesicles.

Lamellar vesicle samples were made by combining a surfactant (e.g., behentriamonium chloride (BTAC), sodium dodecyl sulfate (SDS), or cetrimonium bromide (CTAB)) with a high molecular weight alcohol (e.g., cetearyl alcohol (CA) or 1-decanol (DEC)) at 80° C. and mixing the components to create a homogeneous suspension. After cooling and aging, samples were studied on a 7T Varian/Agilent system at 22° C. using a 1D spin-echo sequence in projection mode. Parameters were δ=5 ms, Δ=100 ms, TR/TE=8000/120 ms, and b-values from 0 to 10,000 s/mm. The normalized log-signal intensity as a function of b-value was fit with both a biexponential curve (derived D1, D2 diffusion, and P2 restricted fraction parameters) and a DKI model (derived apparent diffusion, Dₐₚₚ, and kurtosis, K, parameters constrained to a valid description range of b-value<3/(Kₐₚₚ× Dₐₚₚ).

The data collected indicated that lamellar vesicles of CA-BTAC from 0.5 to 5% (w/w) provide regions of relatively free and restricted diffusion. Embodiments provide that Kₐₚₚ values are tuned to a desired range by adjusting the weight percent of CA-BTAC suspensions contained in the sample. Vesicles made from DEC-CTAB are more porous that those made from CA-BTAC, which results in similar Dₐₚₚ but different Kₐₚₚ values. Sonication of a CA-SDS sample reduces the micron sized vesicles (determined by Coulter counter (not shown)) to 100s of nanometers and hence reduces the volume fraction of restricted water molecules from 28% to 0.8%. Constructing the CA-SDS vesicles with a different alcohol:surfactant ratio does not change Dₐₚₚ (e.g., =0.55 10⁻³ mm²/s) but does reduce Kₐₚₚ from 0.99 to 0.68. Varying the weight percent of CA-BTAC generates a nearly linear increase in Kₐₚₚ (Table 2). In addition, Dₐₚₚ also decreases with increasing CA-BTAC concentration. In some embodiments, independent control of Kₐₚₚ and Dₐₚₚ is provided by keeping the weight percent of material constant and adjusting either the vesicle size or adjusting the vesicle compositions (e.g., 2:1 vs 5:1 w/w % CA:SDS, Table 2).

TABLE 2
Vesicle data

| | Conc. | Biexponential model | | | Kurtosis model | |
|---|---|---|---|---|---|---|
| Sample | wt % | D1(×10³)* | D2(×10⁶) | P2(%) | Dₐₚₚ (×10³) | Kₐₚₚ |
| CA- | 0.5% | 1.80 | 1.1 | 13 | 1.59 | 0.76 |
| BTAG | 1% | 1.59 | 1.61 | 28 | 1.08 | 1.22 |
| | 2% | 1.73 | 1.56 | 39 | 1.02 | 1.78 |
| | 3% | 1.45 | 3.37 | 54 | 0.64 | 2.81 |
| | 4% | 1.06 | 4.33 | 64 | 0.36 | 4.12 |
| | 5% | 0.81 | 3.89 | 68 | 0.24 | 4.50 |
| DEC- | 1% | 1.55 | 35.1 | 27 | 1.11 | 1.14 |
| CTAB | | | | | | |
| CA- | 1:2 1.6% | 0.78 | 56.7 | 28 | 0.55 | 0.99 |
| SDS | native | | | | | |
| | 1:5 1.6% | 0.70 | 103 | 21 | 0.56 | 0.68 |
| | native | | | | | |
| | 1:2 1.6% | 1.35 | 12.0 | 0.8 | 1.30 | −0.05 |
| | sonicated | | | | | |

*Diffusion constants in mm²/s
95% CIs are less than 10% of estimated value

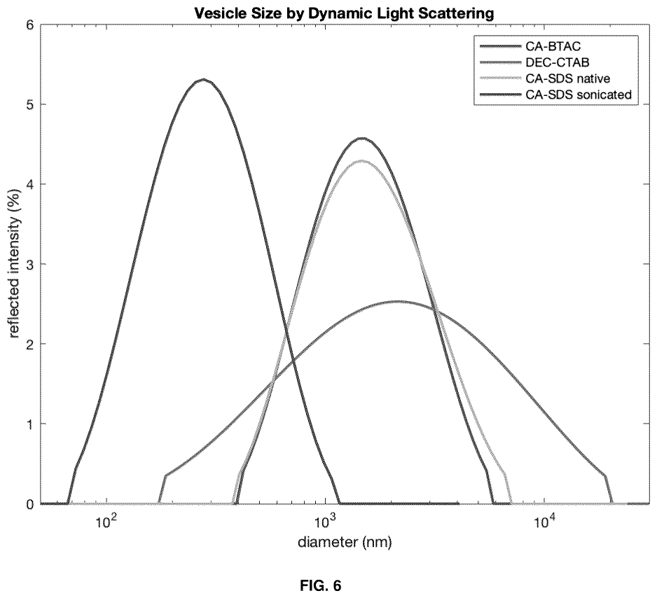

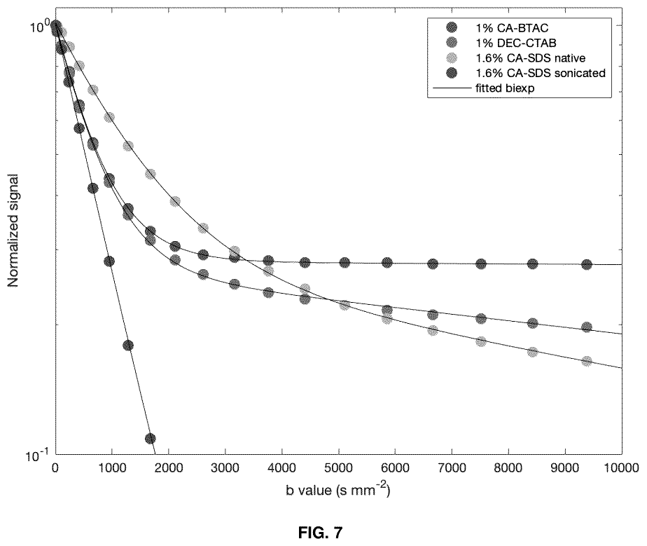

Further, dynamic light scattering measurements indicated micron-sized vesicles formed in water from cetearyl alcohol and BTAC, decanol and CTAB, and from unsonicated cetearyl alcohol and SDS. Dynamic light scattering measurements indicated 200-nm sized vesicles for sonicated cetearyl alcohol and SDS (FIG. 6). Diffusion measurements of similar samples indicated that the larger vesicles exhibited a significant restricted diffusion component whereas the sonicated cetearyl alcohol-SDS samples had a much smaller restricted diffusion component (FIG. 7).

### Example 2

As described herein, the chemical composition of lamellar lipid vesicles determines particle diameter and therefore the pool size of a restricted diffusion component. Accordingly, during the development of embodiments of the technology described herein, experiments were conducted to control DKI phantom parameters by changing vesicle size in CA-CTAB phantoms. During these experiments, data were collected that indicated that phantoms made at 1% by weight solid using cetostearyl alcohol and cetrimonium bromide with varying molar ratios had different apparent diffusion constants and kurtosis values. In addition, vesicle sizes were measured by dynamic light scattering and electron microscopy.

As described herein, phantoms having known MR parameters are needed, e.g., to validate quantitative measurements in multisite MRI studies (see, e.g., Malyarenko (2019) “Multicenter Repeatability Study of a Novel Quantitative Diffusion Kurtosis Imaging Phantom” Tomography 5 (1): 36-43, incorporated herein by reference). In particular, changing the concentration of lamellar lipid vesicles creates diffusion phantoms with tunable apparent diffusion and kurtosis values, Dₐₚₚ and Kₐₚₚ (see, e.g., Swanson (2019) “Tunable diffusion kurtosis in lamellar vesicle suspensions toward development of quantitative phantom surrogate of tumor microenvironment” Proc. Intl Soc Mag Reson Med 2019, Abstract 3632, Montreal, Quebec, Canada, incorporated herein by reference).

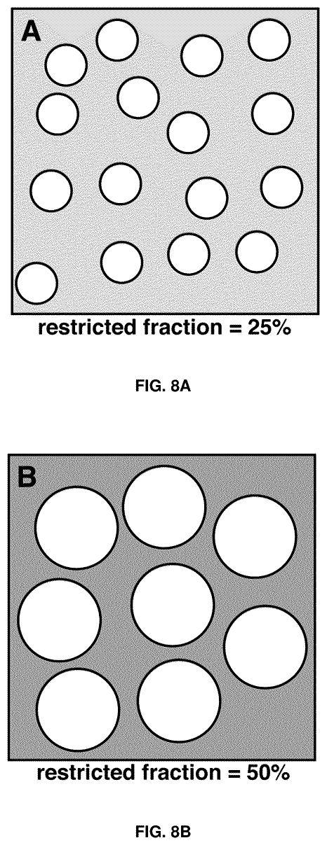

During the development of embodiments of the technology described herein, experiments were conducted to vary the molar ratio of lipid to surfactant to change vesicle diameter and restricted diffusion fraction, thus leading to changes in Dₐₚₚ and Kₐₚₚ values. Given a constant surface area (e.g., a constant weight fraction of molecules), the volume enclosed by vesicles increases linearly with vesicle diameter (see, e.g., FIG. 8, which is a schematic drawing showing a two-dimensional analogy to the three-dimensional vesicle compositions described herein. FIG. 8 thus demonstrates how increasing diameter increases restricted fractions of water molecules). In some embodiments, the vesicles provide a basis for a quantitative diffusion phantom and future MR studies of nanoscale domains.

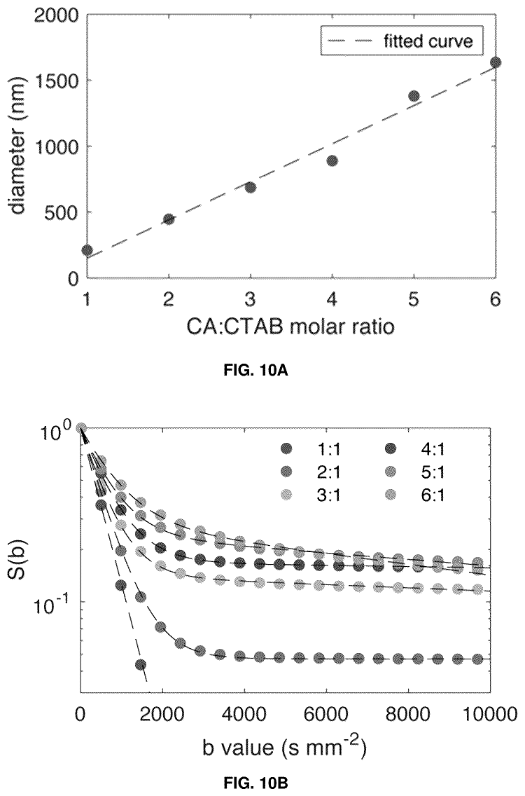

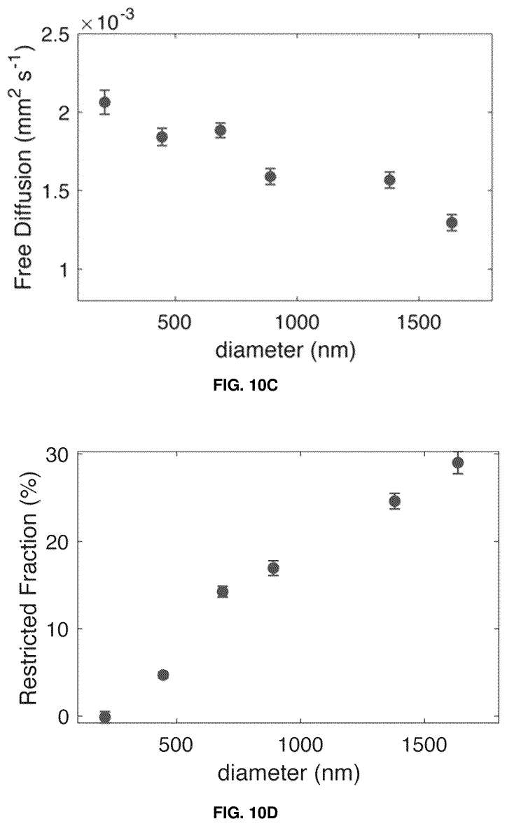

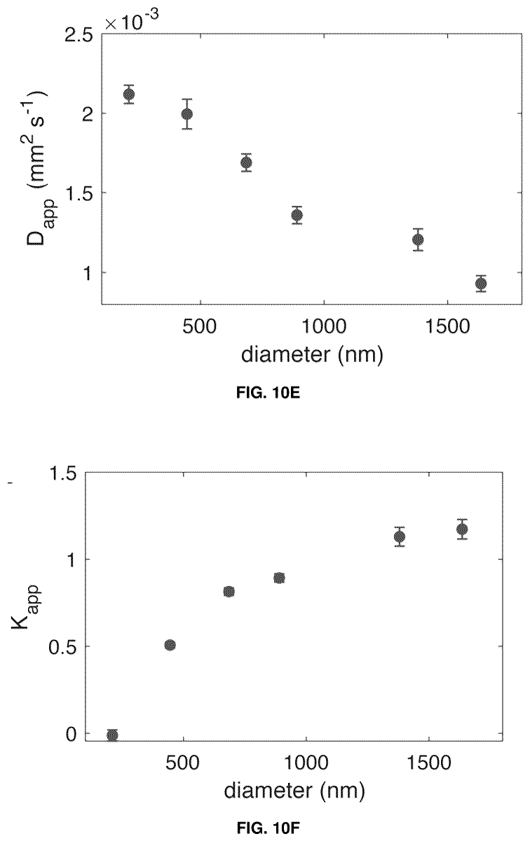

In particular, experiments were conducted using vesicles produced using a constant 1% (w/w) solids-to-water of cetostearyl alcohol (CA) and cetrimonium bromide (CTAB) and a CA:CTAB molar ratio varying from 1:1 to 6:1. CTAB was dissolved in hot water (80° C.) and melted CA was added to form vesicles by stirring and cooling below the lipid transition temperature. All samples were filtered with through a 5-micron polycarbonate filter. Dynamic light scattering was performed at 1 mg/ml concentration and cumulant particle diameter, <d>, and polydispersity index, pi, were determined. Transmission electron microscopy (TEM) was performed on representative samples to visualize vesicle shape and dimensions. NMR water signals were recorded on an Agilent 700 MHz system using a dual stimulated echo experiment to suppress convective flow with b-values up to 10,000 s mm-2. Diffusion time A was 100 ms and gradient duration 8 was 2 ms. The diffusion NMR signal was fitted in Matlab to a biexponential model with free and restricted diffusion, and to a kurtosis model with apparent diffusion and kurtosis values at appropriate b-values (see, e.g., Malyarenko, supra, and Swanson, supra, each of which is incorporated herein by reference). The biexponential model is given as

$$S ( b ) = {p}_{f} exp ( - b {D}_{f} ) + {p}_{r} exp ( - b {D}_{r} )$$

where p_{f} and pᵣ represent the percentage of free and restricted water molecules, respectively, and D_{f} and Dᵣ are the free and restricted water diffusion constants, respectively. The kurtosis model is given by Equation 1 with parameters as defined above (see, e.g., Malyarenko, supra, and Swanson, supra, each of which is incorporated herein by reference). Fitted results are shown in FIG. 10C and FIG. 10D for the biexponential model and FIG. 10E and FIG. 10F for the kurtosis model with error bars representing a 95% confidence interval.

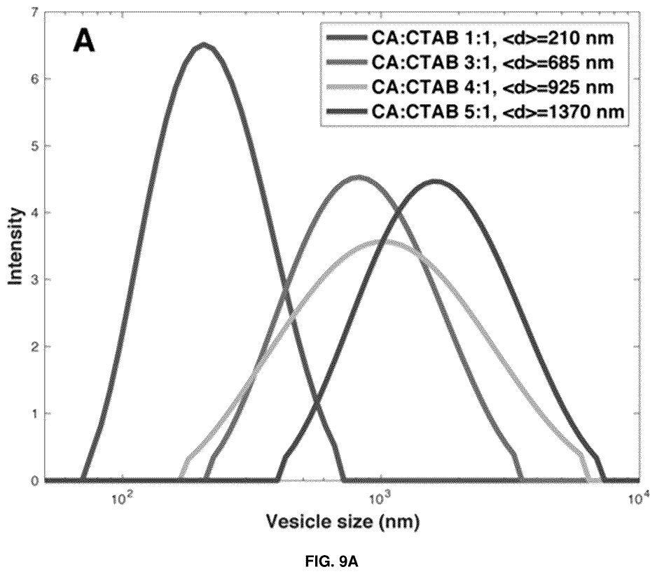

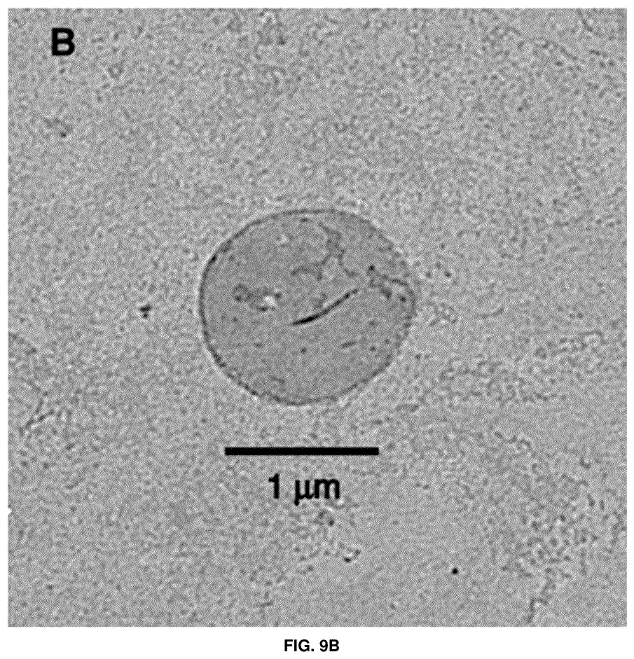

The data collected indicated that the ratio of lipid to surfactant systematically controls vesicle diameter. The diameter of vesicles as measured by dynamic light scattering (DLS) varied from 210 nm to 1380 nm (FIG. 9A). Representative vesicles were detected to be spherical by TEM (FIG. 9B). Vesicle diameter was proportional to CA:CTAB ratio (FIG. 10A) and a biexponential model of diffusion comprising free and restricted components fit all data well (FIG. 10B). Free diffusion, D_{f}, decreased from approximately 0.002 to 0.0015 mm² s⁻¹ in the biexponential model (FIG. 10C) and the apparent diffusion Dₐₚₚ changed from 0.0021 to approximately 0.0009 mm² s⁻¹, indicating differences in the models. More importantly, the restricted fraction in the biexponential model was proportional to the vesicle diameter (FIG. 10D). Kurtosis value (Kₐₚₚ) changed from 0 for the 210-nm vesicles to 1.17 for the 1640-nm vesicles (FIG. 10F). In sum, DLS and NMR (7T) diffusion studies of cetearyl alcohol (CA) and cetyl trimethyl ammonium bromide (CTAB) vesicle-based quantitative kurtosis phantom (at 21° C.) indicated diffusion parameter tuning within tissue-relevant ranges by changing vesicle sizes. Vesicle diameter increased linearly from 210 nm to 1680 nm as CA:CTAB molar ratio varies from 1:1 to 6:1. In kurtosis model, for molar ratio above 2:1, apparent diffusivity, Dₐₚₚ, decreases approximately linearly (from 2 to 0.9×10⁻³ mm²/s) as a function of vesicle diameter and kurtosis, Kₐₚₚ, increases approximately with the square root (from 0.5 up to 1.2). Diffusion was nominally mono-exponential (K=0) for vesicle sizes below 250 nm.

The data collected during the experiments indicated that controlling vesicle diameter provides control of phantom diffusion properties. Accordingly, the technology provides a quantitative diffusion phantom having specified values of Dₐₚₚ and Kₐₚₚ. These vesicles have polydispersity indices between 0.2 and 0.3, with the larger vesicles becoming more disperse. In some embodiments, polydispersity is reduced further by passing the vesicles through appropriately sized polycarbonate filters.

The data collected indicated that the 1% (w/w) samples had low viscosity, flowed easily, and did not trap air or bubbles, in contrast to conventional technologies gels or samples of higher viscosity. The samples tested in these experiments had relatively large values of Dₐₚₚ, which could be reduced with a polymer such as PVP or methylcellulose, while leaving the Kₐₚₚ unaffected (see, e.g., Pierpaoli et al., editors (2009) “Polyvinylpyrrolidone (PVP) water solutions as isotropic phantoms for diffusion MRI studies” Proc. Intl Soc Magn Reson Med; Honolulu, Hawaii, incorporated herein by reference). In some embodiments, samples comprising vesicles having a known diameter find use in studying novel diffusion MRI experiments tuned to a particular vesicle size or microenvironment (see, e.g., Le Bihan D. Mousion (1995) “Tissue microdynamics and microstructure” NMR Biomed. 8 (7): 375-386, incorporated herein by reference. As indicated by the experiments conducted herein, lamellar vesicles provide a molecular platform for quantitative diffusion phantoms. They are stable, easily generated, and can be made at large volumes with tunable diffusion parameters. Accordingly, embodiments of the technology find use in testing (e.g., benchmarking) MRI systems (e.g., in multicenter clinical trials) and providing well characterized nanoscale platforms for future development of novel MRI pulse sequences.

### Example 3

During the development of embodiments of the technology described herein, experiments were conducted to control Dₐₚₚ and Kₐₚₚ independently by inclusion of PVP. Experiments were conducted in which test phantom samples were produced comprising CA-BTAC (Tresemme-based water solutions) or CA-BTAC2 (comprising CA, PDMS, SAPDA, BTAC, KCl, LAC, and EDTA) and PVP. The data indicated that PVP provided for the independent manipulation of Dₐₚₚ for CA-BTAC samples and Kₐₚₚ for CA-BTAC2 samples (Table 3).

Partial charges on the polyvinylpyrrolidone (PVP) allow coating of the positively charged CA-BTAC and CA-BTAC2 vesicles. At low 1.6% concentration, PVP coats the vesicles and imparts increased kurtosis in 2.3% CA-BTAC2. CA-BTAC has additional polymers such as gelatin that create additional coatings and further increase kurtosis. Specifically, the kurtosis values of 2.3% CA-BTAC formulations increase from 0.96 with no polymers to 1.6 with PVP and to 2.08 with PVP plus other polymers. When PVP concentration is increased to 10%, additional polymer is in solution creating excluded volume and reducing Dₐₚₚ constant with minimal effect on Kₐₚₚ.

TABLE 3
Dₐₚₚ and Kₐₚₚ for BTAC and BTAC2 phantoms ± PVP

| | | Biexponential model | | | Kurtosis model | |
|---|---|---|---|---|---|---|
| | Conc. | D1 | D2 | P2 | Dₐₚₚ | |
| Sample | wt % | (×10³)* | (×10⁶) | (%) | (×10³) | Kₐₚₚ |
| CA-BTAC2 | 2.3% | 1.0 | 34 | 19.9 | 0.85 | 0.96 |
| | PVP 0% | | | | | |
| CA-BTAC2 | 2.3% | 1.3 | 13 | 35.1 | 0.85 | 1.60 |
| | PVP 1.6% | | | | | |
| CA-BTAC | 2.3% | 1.5 | 4.5 | 43.1 | 0.83 | 2.08 |
| CA-BTAC | 0.5% + | 1.80 | 1.1 | 13 | 1.98 | 0.61 |
| | 10% PVP | | | | 1.56 | 0.62 |
| | 1.0% + | 1.59 | 1.61 | 28 | 1.51 | 0.89 |
| | 10% PVP | | | | 1.26 | 0.79 |

*Diffusion constants in mm²/s
95% confidence intervals are less than 10% of estimated value

The formulation for CA-BTAC (based on Tresemme solutions) comprised water, cetearyl alcohol (CA), polydimethylsiloxane (PDMS), stearamidopropyl dimethylamine (SAPDA), behentrimonium chloride (BTAC), gelatin (GEL), potassium chloride (KCl), lactic acid (LA), disodium EDTA (EDTA), cetrimonium bromide (CTAB), PVP, and polysorbate 20 (PS20). A 10% (wt/wt) suspension of formulation CA-BTAC was made by heating an appropriate amount of water to 80° C. and adding hydrophilic components (GEL, KCl, LA, EDTA, PVP, PS20) to the hot water. Hydrophobic ingredients (CA, PDMS, BTAC, CTAB) were melted together at 80° C. and added to the hot water solution. pH was measured and adjusted with LAC to provide a pH less than or equal to 6.0. CA-BTAC2 comprised 61% CA, 17% PDMS, 13% SAPDA, and 9% BTAC. Components were melted at 80° C. to form a wax-like paste. 1.6 g of the paste was added to 100 ml of hot water at 80° C. and dispersed. 40 mg of EDTA was added the solution. Lactic acid was added to provide a pH less than 6.0. CA-BTAC2 was studied with and without the addition of 40 kD PVP at 1.6% (w/w).

### Example 4

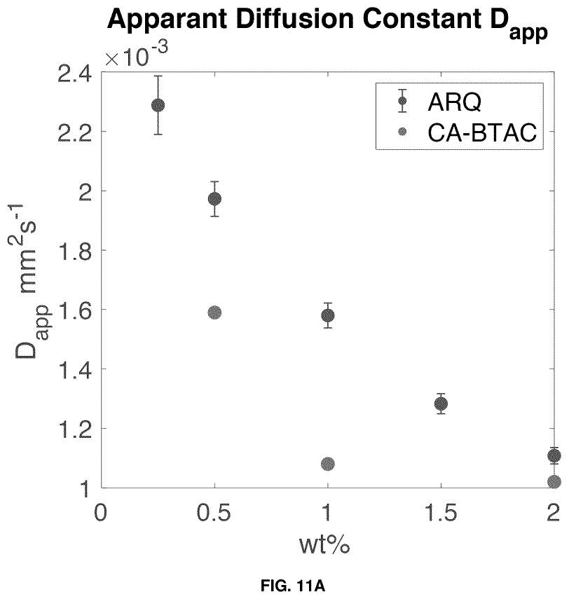

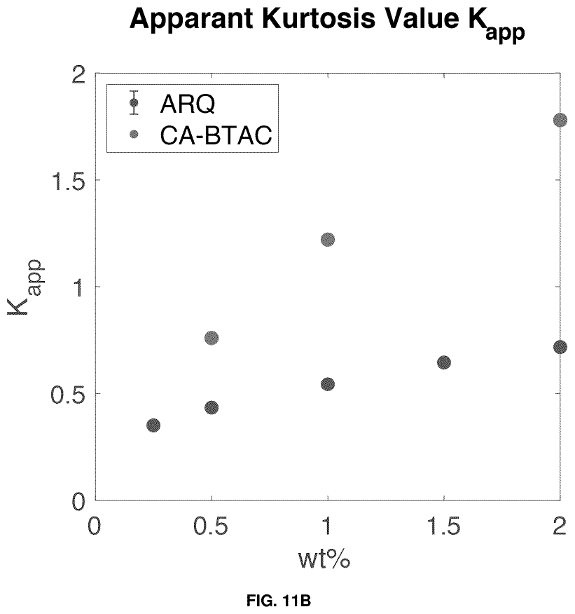

During the development of embodiments of the technology provided herein, experiments were conducted to test phantoms comprising dual chain quaternary ammonium compound (QAC) (e.g., dimethyldioctadecylammonium bromide, dimethyldioctadecylammonium chloride, or di(hydrogenated tallow)dimethylammonium chloride, e.g., commercially available as ARQUAD 2HT-75 (“ARQ”) from SIGMA)). Dual chained QAC molecules spontaneously form vesicles with smaller diameter than CA-BTAC and create Dₐₚₚ and Kₐₚₚ values similar to those found in healthy tissue. Lamellar vesicles with desirable properties are formed when ARQ is dissolved in water at concentration between 0.25% and 2%. ARQ samples were constructed by diluting the produce as delivered by Sigma in water to make a 10% by weight ARQ batch. This material was diluted to 0.25%, 0.5%, 1%, 1.5%, and 2% by weight in distilled water The data indicated that the alkyl ammonium surfactant provided a higher Dₐₚₚ range (Table 4; FIG. 11A and FIG. 11B).

TABLE 4
Dₐₚₚ and Kₐₚₚ for quaternary ammonium compound phantoms

| | | Biexponential model | | | Kurtosis model | |
|---|---|---|---|---|---|---|
| | Conc. | D1 | D2 | P2 | Dₐₚₚ | |
| Sample | wt % | (×10³)* | (×10⁶) | (%) | (×10³) | Kₐₚₚ |
| ARQ | 0.25% | 2.1 | 69 | 1.7 | 2.3 | 0.35 |
| | 0.5% | 1.9 | 67 | 3.6 | 2.0 | 0.43 |
| | 1.0% | 1.6 | 67 | 7.3 | 1.6 | 0.54 |
| | 1.5% | 1.4 | 67 | 10.9 | 1.3 | 0.64 |
| | 2.0% | 1.3 | | 14.2 | 1.10 | 0.71 |

*Diffusion constants in mm²/s
95% confidence intervals are less than 10% of estimated value

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

1-69. (canceled)

70. A method for validating diffusion kurtosis imaging (DKI) parameters as quantitative imaging biomarkers, said method comprising:
a) providing a DKI phantom comprising a high molecular weight alcohol, an ionic surfactant, and water;
b) determining a Kₐₚₚ value from diffusion weighted imaging data obtained from magnetic resonance imaging scan of said DKI phantom; and
c) determining quantitative imaging parameters that quantify non Gaussian diffusion and/or multi exponential relaxation of imaging data obtained from magnetic resonance imaging scan of said DKI phantom.

71. The method of claim 70, wherein the ionic surfactant is behentrimonium chloride (BTAC).

72. The method of claim 70, wherein the high molecular weight alcohol comprises one or more alcohols having 10⁻²² carbons.

73. The method of claim 70, wherein the DKI phantom comprises the high-molecular weight alcohol and ionic surfactant in an amount of 0.25% to 20% by weight.

74. The method of claim 70, wherein the Kₐₚₚ value is at least 0.2 to 3.0.

75. The method of claim 70, wherein the DKI phantom is embedded in a gel.

76. The method of claim 70, wherein the DKI phantom further comprises polyvinylpyrrolidone (PVP) at 5% to 20% by weight.

77. The method of claim 70, wherein the DKI phantom further comprises a nonionic surfactant.

78. The method of claim 70, wherein the DKI phantom produces the same Kₐₚₚ over a period of more than two to sixth months.

79. The method of claim 70, further comprising determining Dₐₚₚ, R₂, R₁, and compartment fractions.

80. The method of claim 70, further comprising comparing the Kₐₚₚ value to a previously determined Kₐₚₚ value.

81. The method of claim 70, further comprising comparing the Kₐₚₚ value to a Kₐₚₚ value determined using another magnetic resonance imaging scanner under similar conditions.

82. The method of claim 70, further comprising validating quantitative magnetic resonance imaging biomarkers of tumor response to therapy.

83. The method of claim 70, further comprising validating the precision and accuracy of quantitative magnetic resonance imaging biomarkers of multiple magnetic resonance imaging scanner platforms and model fit algorithms using a common magnetic resonance imaging scan protocol.

84. The method of claim 70, wherein the method provides true parameter values in physiologically relevant ranges.

85. The method of claim 70, further comprising comparing a first magnetic resonance imaging scanner to a second magnetic resonance imaging scanner.

86. The method of claim 70, further comprising refining fit algorithm accuracy and validating a quantitative diffusion model.

87. The method of claim 70, further comprising evaluating and/or calibrating a magnetic imaging resonance scanner.

88. The method of claim 70, wherein the high-molecular weight alcohol comprises the high-molecular weight alcohol and the ionic surfactant in a weight ratio of from approximately 2:1 to 5:1.

89. The method of claim 70, wherein providing the DKI phantom comprises:
i) mixing a plurality of high-molecular weight alcohols, the ionic surfactant, and a nonionic surfactant in water to provide a mixture;
ii) mixing a portion of the mixture in water to provide a suspension comprising 0.5% to 2.5% of the high-molecular weight alcohols, the ionic surfactant, and a nonionic surfactant.
